(19) United States
(12) Patent Application Publication
(10) Pub. No.: US 2025/0352697 A1
(43) Pub. Date: Nov. 20, 2025

(54) DERMIS DERIVED ALLOGRAFTS AND METHODS FOR PREPARING SAME

(71) Applicant: Musculoskeletal Transplant Foundation, Edison, NJ (US)

(72) Inventors: Evangelia Chnari, Scotch Plains, NJ (US); Evan Friedman, Upper Saddle River, NJ (US); Juliana Gullotta, Ossining, NY (US); Todd Nilsen, Thousand Oaks, CA (US); Rachael Drucker, Aberdeen, NJ (US); Marc Long, Monmouth Junction, NJ (US)

(21) Appl. No.: 19/210,680

(22) Filed: May 16, 2025

Related U.S. Application Data
(60) Provisional application No. 63/648,330, filed on May 16, 2024.

(51) Int. Cl. A61L 27/36; A61L 27/56

(52) U.S. Cl. CPC A61L 27/362; A61L 27/56; A61L 2430/34; A61L 2430/40

(57) ABSTRACT

Improved allografts having controlled and consistent properties and useful for soft tissue repair, including breast reconstruction, and other surgical procedures, are disclosed. In one embodiment, an improved acellular dermal matrix (ADM) is produced from a skin sample, where the ADM consists essentially of a single dermal tissue type (e.g., papillary or reticular) and comprises a collagen matrix having substantially uniform density and porosity. In another embodiment, two ADMs are produced from the same dermal tissue type (e.g., papillary or reticular) of the same skin sample, each of which consists essentially of reticular dermis, and comprises a collagen matrix having substantially uniform density and porosity. All such improved ADMs are suitable for use in breast reconstruction and other tissue repair and modification surgery procedures.

### CROSS-REFERENCE TO RELATED APPLICATIONS

The present application claims the benefit of U.S. Provisional Application No. 63/648,330, filed May 16, 2024, the entire disclosure of which is incorporated by reference herein.

### FIELD OF THE INVENTION

The present invention relates generally to improved dermal matrices made from decellularized dermal tissues, and in particular, selected layers of dermal tissue or portions thereof, as well as the use of such dermal matrices for soft tissue repair, including breast reconstruction and other soft tissue repair and modification surgical procedures.

### BACKGROUND OF THE INVENTION

Mammalian dermal tissue, including human dermal tissue, has been widely accepted and successfully used in various surgical procedures for decades. For example, acellular dermal matrices (“ADMs”) derived from mammalian dermal tissue, including human allograft dermal tissue, are used in the repair of ventral abdominal hernias and other abdominal wall defects, as well as breast repair and reconstruction procedures, and other soft tissue repair treatments including tissue repair and tissue modification, such as, without limitation, plastic surgery procedures.

There are several commercially available versions of such acellular dermal matrices, including allografts derived from decellularized human dermal tissue, which have various properties, including dimensions and biomechanical characteristics, which are selected according to the intended use of the acellular dermal matrices. Such properties include, but are not limited to, mechanical strength (e.g., tensile, burst strength, tear resistance, resistance to suture pull-out, etc.), elasticity, composition (e.g., types and relative amounts of collagen and other extracellular matrix proteins including elastin, glycosaminoglycans and hyaluronic acid), suppleness, flexibility, stiffness, thickness, surface area, porosity, density, types and quantities of growth factors and other proteins, among others of interest and importance.

The properties of acellular dermal matrices and grafts including them may be controlled and determined, at least in part, by the particular processing techniques applied to dermal tissue to produce the matrices. Such properties may also be controlled and determined, at least in part, by the selection and isolation of specific portions or layers of dermal tissue to include in the acellular dermal matrices and grafts including them. In many cases, it is a combination of these approaches that is applied to produce acellular dermal matrices and grafts including them which have the desired properties.

Methods for the production of improved dermal matrices which are useful as graft material for any of several possible treatment procedures are continually being developed and refined. With the relatively recent emergence of a better and clearer understanding of the properties possessed by different portions, layers, and combinations thereof, of dermal tissue, as well as more reliable and precise methods and equipment for reshaping and isolating selected portions, layers, or combinations thereof, of dermal tissue, improved methods for producing such dermal matrices and grafts including them are being developed, as well as the more precisely designed and effective (improved) dermal matrices and grafts produced thereby. Certain improved production methods and the improved dermal matrices and grafts resulting from those methods are described herein.

### SUMMARY OF THE INVENTION

A dermal tissue form is provided for use as graft material in surgical procedures, and comprises a portion of dermal tissue which has been selected and precisely separated and isolated to consist of a single dermal tissue type. In some embodiments, two or more such dermal tissue forms are produced from the same skin sample, each consisting essentially of a single dermal tissue type which is the same type of dermal tissue for all matrices produced from the same skin sample. For example, in some embodiments in which a first dermal tissue form and a second dermal tissue form are produced from the same skin sample, and the first dermal tissue form consists essentially of reticular dermis, then the second dermal tissue form also consists essentially of reticular dermis.

A dermal tissue form is provided which is useful in surgical procedures and is derived from a donor skin tissue which comprised (a) an epidermis; (b) a dermis underlying the epidermis, the dermis including a papillary dermis adjacent the epidermis, a reticular dermis distal to the epidermis, a papillary-reticular dermis interface between the papillary dermis and reticular dermis; (c) and optionally a hypodermis underlying the reticular dermis of (b) the dermis and containing adipose tissue, wherein the reticular dermis has an initial reticular thickness defined between a first reticular defining plane and a second reticular defining plane, each of which is parallel to a reference plane which passes through the skin sample in an orientation approximately parallel with the papillary-reticular interface. The dermal tissue form consists essentially of reticular dermis and comprises: a first exposed surface formed by reticular dermis, an opposite second exposed surface formed by reticular dermis, a desired thickness between the first and second exposed surfaces, and uniform density and uniform porosity between the first and second exposed surfaces, wherein the dermal tissue form promotes rapid and efficient cellular ingrowth and tissue ingrowth substantially equally from either the first or second exposed surfaces upon implantation.

Also provided is a pair of dermal tissue forms, each of which is useful in surgical procedures and consists essentially of reticular dermis, and both of which are derived from a single donor skin tissue which comprised (a) an epidermis; (b) a dermis underlying the epidermis, the dermis including a papillary dermis adjacent the epidermis, a reticular dermis distal to the epidermis, a papillary-reticular dermis interface between the papillary dermis and reticular dermis; (c) and optionally a hypodermis underlying the reticular dermis of (b) the dermis and containing adipose tissue, wherein the reticular dermis has an initial reticular thickness defined between a first reticular defining plane and a second reticular defining plane, each of which is parallel to a reference plane which passes through the skin sample in an orientation approximately parallel with the papillary-reticular interface. Additionally, a first dermal tissue form of the pair comprises: a first exposed surface formed by reticular dermis, an opposite second exposed surface formed by reticular dermis, a first desired thickness between the first and second exposed surfaces, and uniform density and uniform porosity between the first and second exposed surfaces, and a second dermal tissue form of the pair comprises: a third exposed surface formed by reticular dermis, an opposite fourth exposed surface formed by reticular dermis, a second desired thickness between the third and fourth exposed surfaces, and uniform density and uniform porosity between the third and fourth exposed surfaces. Each of the pair of dermal tissue forms promotes rapid and efficient cellular ingrowth and tissue ingrowth substantially equally from either the first or second exposed surfaces upon implantation.

In some embodiments, at least one of the pair of dermal tissue forms is a particulate dermal tissue form and comprises particles, fibers, or both. In some embodiments, the particulate tissue form is combined with one or more biocompatible carriers.

A method is also provided for producing at least a pair of dermal tissue forms, comprising at least a first dermal tissue form and a second dermal tissue form, each of which is useful for soft tissue repair in surgical procedures and consists essentially of reticular dermis, and all of which are derived from a single donor skin sample, the method comprising the steps of: providing a donor tissue including a skin sample having: (a) an epidermis; (b) a dermis underlying the epidermis, the dermis including a papillary dermis adjacent the epidermis, a reticular dermis distal to the epidermis, and a papillary-reticular dermis interface between the papillary dermis and reticular dermis; and (c) optionally a hypodermis underlying the reticular dermis and containing adipose tissue, wherein the reticular dermis has an initial reticular thickness defined between a first reticular defining plane and a second reticular defining plane, each of which is parallel to a reference plane which passes through the skin sample in an orientation approximately parallel with the papillary-reticular interface. The method further comprises: making at least three planar cuts, which comprise a first planar cut, a second planar cut, and a third planar cut, each of which is made into the reticular dermis of the skin sample and is substantially parallel to other planar cuts, or substantially parallel to the reference plane, or both; isolating and recovering a first isolated portion of dermal tissue having a first thickness between a first exposed surface and an opposite second exposed surface thereof, and a second isolated portion of dermal tissue having a second thickness between a third exposed surface and an opposite fourth exposed surface thereof, each of the first isolated portion and the second isolated portion consists essentially of reticular dermis, wherein the sum of the first thickness and the second thickness is no greater than the initial reticular thickness of the reticular dermis of the single skin sample; and producing: the first dermal tissue form using the first isolated portion of dermal tissue, wherein the first dermal tissue form has a desired first thickness equal to the first thickness of the first isolated potion of dermal tissue, and producing the second dermal tissue form using the second isolated portion of dermal tissue, wherein the second dermal tissue form has a desired second thickness equal to the second thickness of the second isolated potion of dermal tissue.

The step of producing the first and second dermal tissue forms comprises performing one or more processing techniques on each of the first and second isolated portions of dermal tissue, wherein the one or more processing techniques performed on the first isolated portion is different or the same as those performed on the second isolated portion.

In some embodiments, the first thickness of the first isolated portion of dermal tissue is no more than about 80% of the initial reticular thickness and the second thickness of the second isolated portion of dermal tissue is at least 20% of the initial reticular thickness.

### BRIEF DESCRIPTION OF THE DRAWINGS

The present invention will be further explained with reference to the attached drawings, wherein like structures are referred to by like numerals and/or letters throughout the several views. The drawings shown are not necessarily to scale, with emphasis instead generally on illustrating the principles of the presently disclosed and contemplated invention.

FIG. 1 is a schematic diagram showing a section of human skin and the various components thereof;

FIG. 2 is a schematic perspective view of an ADM positioned, during breast reconstruction surgery, in a pre-pectoral position and secured to the pectoral (breast) muscle of a patient according to an exemplary use of the improved ADM described herein;

FIG. 3 is a schematic cross-sectional view of the breast and ADM shown in FIG. 2, with the ADM in a pre-pectoral position, forming a pocket having an implant held therein, so that the ADM provides a sling for conforming to and supporting the patient's skin flap;

FIG. 4 is a schematic diagram of the section of human skin of FIG. 1 showing several reference features of the skin for orientation and understanding of the how to determine where to make cuts into the skin sample according to several embodiments of the method of the present invention;

FIG. 5 is a schematic diagram of the section of human skin of FIG. 4 illustrating the cutting steps performed on the skin according to an exemplary embodiment of the present invention which produces an improved acellular dermal matrix consisting essentially of a single dermal tissue type;

FIG. 6 is a schematic diagram of the section of human skin of FIG. 4 illustrating the cutting steps performed on the skin according to an exemplary embodiment of the present invention which produces at least two improved acellular dermal matrices, both of which consist essentially of a single dermal tissue type, which is the same for both matrices.

FIG. 7 provides histology images (magnification 4× and 2×), each produced with saturation of 4000% and sharpness of 50%) of three improved ADMs produced from skin samples recovered from three different donors, each improved ADM having uniform cross-sectional density and porosity and produced by a method comprising cutting a skin sample two times as shown in FIG. 5; and

FIG. 8 provides histology images (magnification 4× and 2×), each produced with saturation of 4000% and sharpness of 50%) of three improved ADMs produced from skin samples recovered from three different donors, each improved ADM having uniform cross-sectional density and porosity, and being one of a pair of improved ADMs produced from a single skin sample by a method comprising cutting the skin sample three times as shown in FIG. 6.

### DETAILED DESCRIPTION OF THE INVENTION

Detailed embodiments of the present invention are disclosed herein. It should be understood that the disclosed embodiments are merely illustrative of the invention that may be embodied in various forms. Furthermore, the invention described and contemplated herein generally relates to acellular dermal matrices and grafts including them which are useful for the repair of soft tissue defects.

Various embodiments of the methods and improved ADMs produced thereby are described hereinafter as starting with or derived from human dermal tissue and methods for their processing to produce acellular dermal allografts, or ADMs. However, other mammalian sources for the dermal tissue are just as suitable as the starting materials, including without limitation, ovine, porcine, bovine, equine, canine, feline, rodent, and other mammalian dermal tissue sources.

In addition, various embodiments of the invention are described hereinafter for isolating portions of deep dermal tissue from the reticular dermis RD layer of skin to produce one or more ADMs consisting essentially of reticular dermis RD. However, it is also contemplated that the methods and ADMs described and contemplated herein could also accurately and consistently isolate portions of other dermal tissue, such as portions of the papillary dermis PD to produce improved ADMs consisting essentially of papillary dermis PD.

Moreover, each of the examples described and provided herein in connection with the various embodiments of the invention is intended to be illustrative, and not restrictive. Further, the figures are not necessarily to scale, and some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as examples for teaching one skilled in the art to variously employ the present invention.

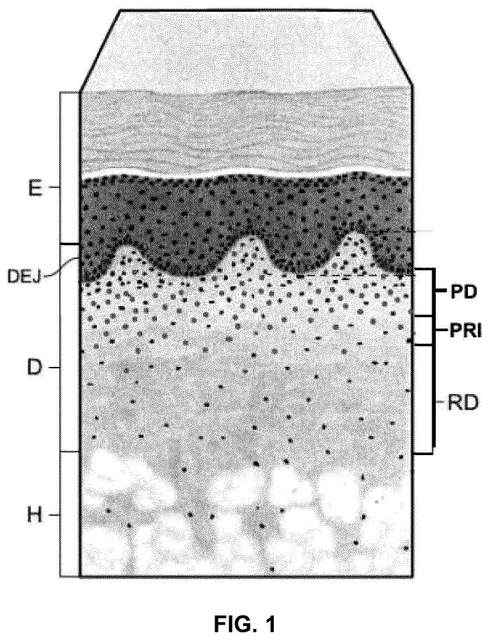

Current commercially available (i.e., existing) acellular dermal matrices (“ADMs”) which are useful as grafts for use in surgical repair and reconstructive procedures include, for example, FlexHD® Structural™ ADM, which is marketed by Musculoskeletal Transplant Foundation (Edison, NJ), as well as AlloDerm® ADM and AlloDerm® Ready to Use (“RTU”) ADM, both of which are marketed by LifeCell Corporation (Branchburg, NJ). The nature and composition of the dermal tissue from which these existing ADMs are derived is explained with reference briefly to FIG. 1, which illustrates the microstructure of human skin.

Human skin, as illustrated in FIG. 1, is recovered from either live or deceased donors, after receiving consent from the individual donor or donor's family. The human skin is made of several layer-like components, which are approximately but not necessarily clearly visually and structurally delineated at their interfaces. Referring to FIG. 1, the layer-like components include the outer-most epidermis E, and the dermis D, which lies beneath the epidermis. The hypodermis H (also sometimes referred to as the subcutis) lies beneath the dermis D, but is not generally considered part of the skin. Rather, the hypodermis H contains adipose and often also some muscle tissue. The dermis D itself includes the papillary dermis PD, which lies adjacent the epidermis E, and the reticular dermis RD, which lies between the papillary dermis PD and the hypodermis H. The papillary-reticular dermis interface PRI, lies between the papillary dermis PD and the reticular dermis RD. The dermis-epidermis junction (“the DEJ”) lies between the papillary dermis PD and epidermis E.

Processes for producing the above-mentioned existing ADMs from a full thickness skin sample (as shown in FIG. 1) generally involve removing the epidermis E (e.g., by a chemical process that causes the epidermis to slough off, leaving an uneven surface), thereby exposing the DEJ that was adjacent to the now absent epidermis E. Beneath the DEJ lies the papillary dermis PD, the papillary-reticular dermal interface PRI, the reticular dermis RD, and possibly the hypodermis H.

When the skin sample does include at least some hypodermis H remaining adjacent to the reticular dermis RD, to produce the ADM, the hypodermis H is often also removed, sometimes along with some of the “deep” portion of the reticular dermis RD which is adjacent the hypodermis H. Removing the hypodermis H from the skin sample is generally accomplished by cutting or slicing (e.g., making a planar cut) through the deep portion of the reticular dermis RD, or even possibly through the interface where the reticular dermis RD and hypodermis H meet.

The dermal portion of the skin sample that is recovered and isolated to form existing ADMs, as described above, generally includes the DEJ, the entire papillary dermis PD and at least part of the reticular dermis RD. Furthermore, the resulting isolated dermal portion of the skin sample essentially lacks the epidermis E, the hypodermis H., and optionally also a portion of the deep portion of reticular dermis RD. The aforesaid isolated dermal portion (i.e., including the DEJ, papillary dermis PD layer, and at least part of the reticular dermis RD layer) is subjected to one or more processing techniques, including without limitation, decellularizing and aseptic processing, to produce an ADM which meets sterility testing requirements.

The microstructure of the papillary dermis PD layer is not uniform. More particularly, the papillary dermis PD transitions from higher collagen density in its upper, more superficial (“epidermal”) portion, to lower collagen density in its lower, deeper (“dermal”) portion. The lower collagen density continues into papillary-reticular interface PRI and reticular dermis RD. This means that the dermal portion of the papillary dermis PD is more porous than the epidermal portion of the papillary dermis PD. The entire papillary dermis PD layer has often been included in the above described existing ADMs, sometimes along with the papillary-reticular interface PRI and even some reticular dermis RD.

The aforesaid dual structure of the papillary dermis PD is also a property of the above described existing ADMs, and can be advantageous for repairing ventral abdominal hernias and other abdominal wall defects, as the more densely-packed epidermal portion of such existing ADMs (i.e., incorporating the epidermal portion of the papillary dermis PD) possess the tensile strength and stiffness required for such load-bearing tissue repairs, and the more porous dermal portion of the existing ADMs (i.e., incorporating the dermal portion of the papillary dermis PD, as well as at least a portion of the loosely-packed and porous underlying reticular dermis RD) provide an open collagen structure that promotes vascularization, cellular attachment and tissue ingrowth, at the surgical treatment site. Nevertheless, this dual structure, which may only be visible on a microscopic scale, presents concerns about identifying and maintaining the side orientation of the ADM, i.e., during a surgical procedure.

The above described existing ADMs, and other allograft dermal tissue derived ADMs, have also been used in plastic surgery procedures, including breast reconstruction, where the existing ADM is implanted to function as an internal sling that is draped around a breast implant and/or tissue expander. While the high tensile strength and stiffness of the existing ADMs are important for hernia and abdominal wall repairs, breast reconstruction and some other tissue repair and modification surgery procedures do not involve the degree of load-bearing and other tissue property considerations inherent in hernia and abdominal wall repairs.

Instead, materials used as slings and similar devices in breast reconstruction should possess biomechanical properties that are well-suited to such applications, including predictable suppleness, flexibility and uniform pliability sufficient for such slings to stretch and expand without tearing during tissue expansion (i.e., using breast implant and/or tissue expander). Furthermore, increased and predictable suppleness of such materials should decrease, or even minimize, formation of ripples under the skin, which are often visible at a reconstruction site where less flexible, less supple grafts have been used, thereby providing improved cosmesis of the reconstruction procedure. Suitable materials for breast reconstruction and other reconstruction and plastic surgery procedures should also possess sufficient tensile strength, preclude suture tear-out, both during implantation and expansion through the post-operative phase.

Finally, it would be advantageous for materials used as slings and similar devices in breast reconstruction to provide grafts having top and bottom surfaces with similar physical properties, such as density, porosity, and texture. This would reduce or eliminate the necessity for surgical practitioners to discern and select one side or the other (i.e., top or bottom surfaces) of such grafts when orienting and implanting them in contact with host tissue during surgical procedures. Grafts having both sides (top and bottom surfaces) with similar physical properties should allow rapid and efficient cellular ingrowth equally from either side of the ADM.

More and more, the properties of various different portions and layers of dermal tissue continue to be better understood and defined. This improved knowledge of the relative properties of different portions and layers of dermal tissue, especially the deeper layers such as the reticular dermis RD and the dermal portion of the papillary dermis PD, enables improved selection and control of the properties of ADMs produced from dermal tissue by enabling more informed selection of specific portions and layers of the dermal tissue to include in the ADMs, based on the desired properties of the ADMs and their intended use.

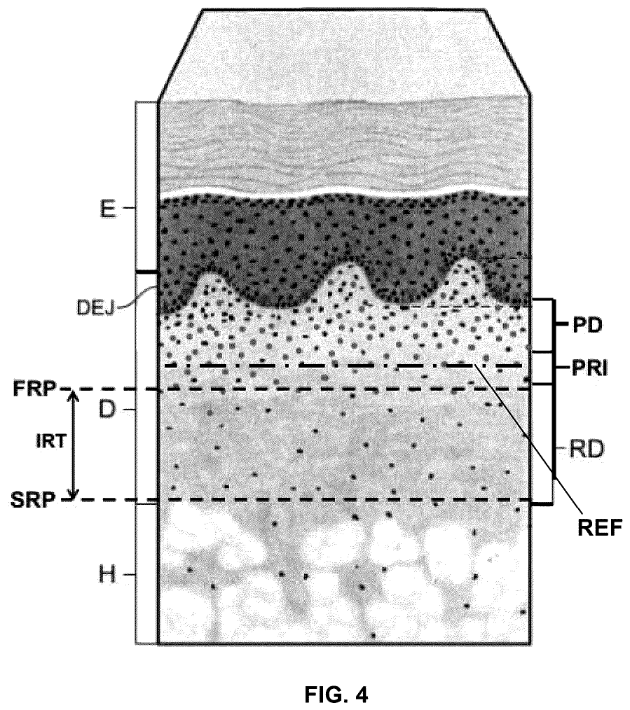

It should be understood that the terms “deep” and “deeper” refer to the location of dermal tissue relative to other dermal tissue portions or layer in a skin sample when the skin is in an epidermis side up orientation as shown in FIGS. 1 and 4. In such an epidermis side up orientation, the epidermal side thereof is generally referred to as the “top” or “upper” portion of a skin sample and the deep and deeper dermal tissues are those located non-adjacent or remote from the epidermal side. For example, the “deep” portion (or dermal side) of the papillary dermis PD layer is located adjacent to the papillary-reticular interface PRI, proximate the reticular dermis RD, and remote from the dermis-epidermis junction DEJ. Similarly, the reticular dermis RD layer is considered a “deep” dermal tissue, but while an upper portion of reticular dermis RD is adjacent and proximate the papillary-reticular interface PRI, a “deeper” portion of the reticular dermis RD is adjacent and proximate to the hypodermis H, and remote from the papillary-reticular interface PRI.

For example, the dermal structure is relatively dense starting at the epidermal side of the skin and moving through the dermis epidermis junction DEJ, into and through the epidermal side of the papillary dermis PD, and becomes progressively looser (less dense), with changes to the types of collagen present as well as to the dermal tissue architecture, as deeper dermal tissue is accessed, i.e., from the dermal portion of the papillary dermis PD, and down through the reticular dermis RD, approaching the hypodermis H. Eventually, a region of the deepest reticular dermis RD is encountered in which adipose is interspersed in the collagen matrix and the dermal tissue structure is so loose that it no longer has sufficient integrity and strength to serve as a supportive three-dimensional scaffold if used as a graft in reconstructive surgery.

A change in the technique for breast reconstruction surgical procedures, from the traditional and prevalent subpectoral technique (breast implant is placed behind and underneath the pectoralis major muscle) to the newer and less invasive pre-pectoral technique (breast implant is placed above and in front of the pectoralis major muscle), brought a shift in the desired and effective properties of ADMs used in in such procedures. For example, larger ADMs were needed for the pre-pectoral breast reconstruction procedure.

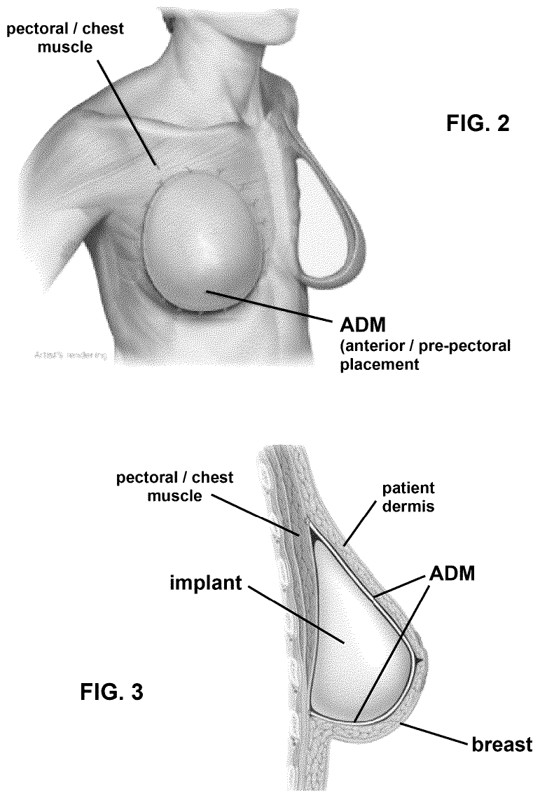

FIGS. 2 and 3 illustrate use and placement of the ADM as a sling to support the patient's skin flap during the placement of an implant (or tissue expander) for breast reconstruction by a pre-pectoral surgical technique. As shown in FIG. 2, the ADM is positioned anteriorly (i.e., in a pre-pectoral location) relative to the pectoral (chest) muscle of a patient and fastened to the pectoral muscle along, or proximate to, the ADM's peripheral edge (e.g., by suturing, stapling, etc.). This positioning and fastening of the ADM forms and provides a pocket or cavity within which a breast implant (or tissue expander) is received and held as shown in the post-operative cut away view of FIG. 3. As can be seen in the drawings of both FIGS. 2 and 3, when placed in a pre-pectoral position, the ADM conforms to the shape of the breast implant (or tissue expander) in its function as a supportive sling.

Applicants surprisingly discovered that, although the deeper dermal layers (e.g., reticular dermis RD) of skin samples are less dense, and therefore may possess lower strength compared to the upper dermal layers (e.g., papillary dermis PD) which are typically included in the above described ADMs, portions of dermal tissue isolated from those deeper dermal layers still possessed the minimum strength required to be effective grafts in breast reconstruction procedures. In other words, it had previously been perceived that deep dermal tissue such as the reticular dermis RD, when isolated without any of the upper dermal tissue such as at least a portion of the papillary dermis PD, might not have sufficient strength to be suitable for use as a graft in breast reconstruction procedures. However, this turned out to be inaccurate.

The discovery was that dermal tissue present deeper in skin samples than that which had been typically and routinely isolated and processed to produce ADMs, also had sufficient biomechanical properties to be included in the isolated portion or layers of dermal tissue which is processed to form improved ADMs described and contemplated herein as suitable for use in breast reconstruction procedures. Furthermore, it had previously been unclear and was doubted whether the deeper dermal tissue of the reticular dermis RD layer contained too much lipid (adipose) to have sufficient integrity and strength to be used in breast reconstruction procedures, as well as whether such isolated portions and layers of deep dermal tissue could be effectively decellularized without losing too much structural integrity and strength to remain useful as a graft in breast reconstruction procedures. The invention described and contemplated herein provides methods which include recovering and isolating deep dermal tissue, followed by processing, to surprisingly produce one or more improved ADMs comprising deep dermal tissue, such as reticular dermis RD, and retaining the properties required for use as a graft in breast reconstruction procedures.

Furthermore, it has been discovered that, in fact, isolated portions of dermal tissue which consist essentially of reticular dermis RD, i.e., even without any of the more dense upper dermal tissue layers (e.g., papillary dermis PD), are suitable and have properties necessary for successful use as grafts in breast reconstruction procedures. Additionally, it was surprisingly discovered that some skin samples have a reticular dermis RD layer having sufficient thickness that it is possible to isolate more than one portion of dermal tissue from the same reticular dermis RD, whereby more than one improved ADM suitable for use in breast reconstruction procedures can be produced from a single skin sample. The foregoing improved understandings provide the potential for increasing, potentially doubling, the number of useful graft material from each skin sample which has a sufficiently thick reticular dermis RD.

Previously, due to the lack of knowledge regarding the thickness of some skin samples, such as those recovered from a donor's back, compared to skin samples recovered from legs and arms, it had been unknown that cutting all skin samples to the same depth or thickness was leaving behind significant portions of deep reticular dermis RD unrecovered and wasted with some skin samples. The above-discussed advancements in the understanding of the true structure and properties of dermal tissue, along with improvements in the preciseness with which portions and layers can be cut and isolated from skin samples and have consistent and predictable properties have enabled the development of the presently described and contemplated methods for making improved ADMs having substantially uniform thickness and sufficient biomechanical strength for use in breast reconstruction, while increasing the yield of such improved ADMs.

Applicants recognized that skin samples obtained from different donors, and even from different regions of the same donor, have different overall thicknesses, as well as different thicknesses for each of the dermal layers, and that this surprisingly and consequently meant that isolating portions of deep dermal tissue having sufficient strength to serve as grafts useful in breast reconstruction procedures cannot rely on cutting every skin sample to the same depth or thickness. Rather, each skin sample should be evaluated and the depth or thickness of the deepest cut to be made into the reticular dermis RD determined to enable removal of the hypodermis H and enough of the deepest reticular dermis RD for the structure of the remaining isolated portion of deep dermal tissue to have sufficient integrity and strength for use as a graft in breast reconstruction procedures. Furthermore, the improved ADMs described and contemplated herein are suitable for use, more generally, for soft tissue repair and reconstruction involving treatments such as, but not limited to, void filling, volumizing, wound care, and soft tissue trauma repair and reconstruction, among others.

The above-described existing ADMs included upper dermal layers, such as the dermis-epidermis junction DEJ, the epidermal side of the papillary dermis PD, as well as deeper dermal layers including the dermal side of the papillary dermis PD and at least a portion of the reticular dermis RD, at least in part because it was believed that such a combination of dermal layers was required to provide ADMs having sufficient strength to be suitable for use in breast reconstruction procedures. Contrary to this conventional wisdom in the relevant art, the methods described and contemplated herein for producing one or more improved ADMs, and the resulting improved ADMs each consisting essentially of an isolated portion of reticular dermis RD were developed based on the discovery that the aforesaid understanding of the nature and properties of dermal tissue layers, especially for the deeper reticular dermis RD, were inaccurate.

Additionally, cutting and measurement techniques and equipment have improved sufficiently to enable more precise separation and isolation of selected portions and layers of dermal tissue for inclusion in ADMs. These provide increased control of the properties of the resulting ADMs by providing accurate and consistent cutting and isolation techniques. For example, the selected preferred portions and layers of dermal tissue (skin sample) can be more precisely isolated and retained, as well as enabling more accurate and complete removal and elimination of unwanted, less advantageous, or even deleterious, portions (e.g., sections, layers) of dermal tissue, to produce improved ADMs. Accurately and consistently cutting skin samples at the desired depth or thickness determined, as described above, based on the maximum depth at which a particular skin sample can be cut to remove the hypodermis H and, optionally, a portion of the deeper reticular dermis RD, enables reliably and consistently isolating and retaining deep dermal tissue having sufficient structural integrity and strength to be suitable for use as an improved ADM graft in breast reconstruction procedures.

Dimensional properties of the produced improved ADMs can also be more precisely designed, such as consistent uniform thickness, which would of course be selected from within the existing dimensions of a particular skin sample being processed, but then is maintained between first and second substantially parallel surfaces of the entire ADM, using the more precise and controllable cutting and measurement techniques and equipment.

Together, the ability to more accurately select portions and layers of dermal tissue based on an improved knowledge of their properties, coupled with the ability to more accurately and precisely separate and isolate those selected portions and layers of dermal tissue and control dimensional properties of the isolated portions of dermal tissue, which is made possible by the availability of improved cutting and measuring equipment, have enabled the development of the invention described and contemplated herein which provides a method for producing more precisely designed ADMs, as well as the ADMs and grafts including them which are more effective grafts for tissue repair and reconstruction surgical treatments.

The improved ADMs described and contemplated herein comprise more precisely selected and isolated portions and layers of dermal tissue with improved structural and biomechanical properties that are more consistent, reliable, and effective for use in breast reconstruction and other reconstructive and plastic surgery procedures. Such properties include, but are not limited to, consistent and predictable suppleness, flexibility, uniform pliability sufficient to stretch and expand without tearing during tissue expansion (i.e., using a breast implant and/or tissue expander), sufficient tensile strength for breast reconstruction and other plastic surgery applications, precise and uniform dimensions (e.g., thickness, surface area), improved handling properties, and substantially uniform porosity that promotes rapid and efficient cellular ingrowth equally from either side of the ADM.

More particularly, the invention described and contemplated herein provides a flexible, pliable, and supportive sheet or patch of acellular dermal matrix or tissue form (improved ADM), useful as a surgical graft or implant, and comprising a section (i.e., one or more portions, layers, or combinations thereof) cut and isolated from a full thickness skin sample (i.e., dermal tissue sample) and subjected to one or more processing techniques. Processing techniques applied to dermal tissue and sections thereof to produce the improved ADMs which more consistently and precisely have desired selected properties and characteristics include, without limitation, one or more of: recovering, isolating, freezing, cleaning, rinsing, soaking, storage, resizing (e.g., cutting, slicing, etc.), decellularizing, contacting with one or more solvents, disinfecting, sterilizing, dehydrating, cross-linking, stabilizing, molding using a mold or other container or support device, as well as repetitions and combinations of these techniques. Resizing and cutting techniques, which retain or discard selected portions or layers of dermal tissue, may also be employed to select and control properties of the resulting acellular dermal matrices and grafts including them. Additionally, further reshaping and modification of the physical form and features may be applied at any point during the production of the improved ADMs and may be applied to one or more portions or regions, or all, of the improved ADMs. Such further reshaping and modifications include, but are not limited to, contouring, perforating, texturizing, punching, die cutting, fenestrating, meshing, slicing, adding one or more slots, openings, troughs, grooves, recesses, indents, etc. of any desired shape(s), and combinations thereof.

In accordance with the invention described and contemplated herein, methods are provided for producing one or more improved ADMs from allograft (i.e., human) skin sample, wherein each of the one or more improved ADMs comprises the same type of dermal tissue derived from the same layer of the skin sample, and each of which comprises the same type of dermal tissue derived from the same single layer or a portion of the same single layer of dermal tissue In embodiments where two or more improved ADMs are produced, each of them comprises a portion of the same type of dermal tissue derived from the same single layer of dermal tissue of the skin sample (i.e., each of the two or more improved ADMs comprise the same type of dermal tissue since they are isolated from the same layer of the skin sample).

For example, without limitation, in some embodiments, one improved ADM is produced which comprises a portion of dermal tissue consisting essentially of substantially an entire single dermal layer which has been isolated from the skin sample, and which may or may not have a substantially uniform thickness as desired. For example, without limitation, the improved ADM may comprise a portion of dermal tissue consisting essentially of substantially an entire papillary dermis PD layer which has been isolated from the skin sample. The improved ADM may, alternatively, comprise a portion of dermal tissue consisting of substantially an entire reticular dermis RD layer which has been isolated from the skin sample. Moreover, the improved ADM may have any of several additional desired and selected properties (e.g., decellularized, substantially uniform thickness, disinfected, desired degree of flexibility and tensile strength, etc.) depending on which of one or more processing techniques are performed and applied to the isolated single dermal layer.

In other embodiments, for example without limitation, two or more improved ADMs are produced, each of which comprises an isolated portion of dermal tissue consisting essentially of a type of dermal tissue which is the same as the type of dermal tissue of the other improved ADM(s). In other words, two or more portions of a single type of dermal tissue (i.e., single dermal layer) have been isolated from the skin sample and each of those two or more isolated portions of dermal tissue forms an improved ADM consisting essentially of a single type of dermal tissue, which is the same type of dermal tissue as the other improved ADMs produced from the skin sample. More particularly, but without limitation, two improved ADMs may be produced, each of which comprises a portion of dermal tissue isolated from the papillary dermis PD of the skin sample and, therefore, each consists essentially of papillary dermis PD. Alternatively, two improved ADMs may be produced, each of which comprises a portion of dermal tissue isolated from the reticular dermis RD of the skin sample and, therefore, each consists essentially of reticular dermis. In still another exemplary embodiment, three improved ADMs may be produced, each of which comprises a portion of dermal tissue isolated from, for example, the reticular dermis RD of the skin sample and, therefore, each of the three isolated portions of dermal tissue (and, therefore, each of the resulting improved ADMs) consists essentially of reticular dermis.

Furthermore, each two or more improved ADMs may have any of several additional desired and selected properties (e.g., decellularized, substantially uniform thickness, disinfected, desired degree of flexibility and tensile strength, etc.) depending on which of one or more processing techniques are performed and applied to each of the two or more isolated portions of dermal tissue (of the same type). Furthermore, each of the two or more isolated portions of dermal tissue (of the same type) may be subjected to the same or different processing techniques and, therefore, each of the resulting two or more improved ADMs may have the same or different selected properties as the other improved ADMs.

Each of the above described ADMs possesses properties that are particularly suited for their use, for example without limitation, as a sling in breast reconstruction, as well as other reconstructive and plastic surgery procedures. For example, but without being limited, the above-described ADMs minimize adhesions and foreign body reactions while promoting vascularization, cellular attachment, and tissue ingrowth at the surgical site.

Compared to the previously described existing ADMs, the presently described and contemplated improved ADMs also possess adequate and possibly improved tensile properties (i.e., strength, pliability, stretchability), equally desirable handling characteristics, and substantially uniform thickness and porosity, which are particularly suitable for their use in breast reconstruction and other plastic surgery applications. The improved ADM also possesses improved suture retention strength, elasticity, and deformability, which are more suited for its intended use in breast reconstruction and other plastic surgery applications than existing ADMs.

The improved ADM is resistant to bacterial colonization and is non-immunogenic, as a result of decellularizing and other processing techniques applied to the isolated portion of dermal tissue which forms the improved ADM.

Generally, and as described in further detail below, the methods described and contemplated herein for producing one or more improved ADMs comprise making two or more planar cuts into a single dermal tissue type (i.e., into a single dermal layer) of a skin sample to produce one or more isolated portions (e.g., layers or sheets) of dermal tissue. Making the planar cuts into the dermal layer may be accomplished by various techniques and devices for example, without limitation, a manual dermatome technique, dissection with a scalpel, using a device having a straight cutting edge, as well as using one or more devices having a cutting component (e.g., blade, razor) with a straight cutting edge and which may or may not be capable of moving either the cutting component or the skin sample.

Each of the resulting isolated portions of dermal tissue consists essentially of the single dermal tissue type. The single dermal tissue type may be reticular dermis RD or papillary dermis PD. In some embodiments, such as when it is desired for each of the one or more improved ADMs to have a substantially uniform thickness, the two or more planar cuts may be substantially parallel to one another, or substantially parallel to a reference plane, or both (e.g., where one performed planar cut is selected as the reference plane).

The reference plane (REF, see FIG. 4) is selected to provide a reference for alignment of all planar cuts and, hopefully, maximize the yield of isolated portions and layers of dermal tissue from any given skin sample. The reference plane may be the first cut made into the skin sample, whereby all subsequent cuts will be aligned substantially parallel to the same reference plane and will, therefore, also be substantially parallel to one another. Alternatively, the reference plane may be approximately aligned one of the natural features of the skin sample such as, without limitation, the dermal-epidermal junction DEJ, the papillary-reticular interface PRI, or even the interface where the reticular dermis RD and hypodermis H meet.

Generally, the method for producing improved ADMs further includes performing one or more processing techniques which are applied to each of the isolated portions or layers of dermal tissue. Processing techniques applied to isolated portions and layers of dermal tissue to produce the improved ADMs include, without limitation, one or more of: recovering, isolating, freezing, cleaning, rinsing, soaking, storage, resizing (e.g., cutting, slicing, etc.), decellularizing, contacting with one or more solvents, dehydrating, cross-linking, stabilizing, molding using a mold or other container or support device, as well as repetitions and combinations of these techniques. Resizing and cutting techniques which retain or discard selected portions or layers of dermal tissue may also be employed to select and control properties of the resulting acellular dermal matrices and grafts including them. The processing techniques applied to each of the isolated portions and layers of dermal tissue do not have to be the same as are applied to others.

Additionally, further reshaping and modification of the physical form and features may be applied at any point during the production of the improved ADMs and may be applied to one or more portions or regions, or all, of the improved ADMs. Such further reshaping and modifications include, but are not limited to, contouring, perforating, texturizing, punching, die cutting, fenestrating, meshing, slicing, adding one or more slots, openings, troughs, grooves, recesses, indents, etc. of any desired shape(s), and combinations thereof.

Furthermore, the method for producing improved ADMs may comprise further post-processing wherein the improved ADM is subjected to one or more additional processing techniques including but not limited to: at least partially dehydrating (e.g., by heating, air drying, lyophilizing, etc.), additional resizing to produce an improved ADM product having desired physical forms, adding or combining the improved ADM with one or more carriers, packaging, and others as determined and desired to produce the improved ADM in a preferred form and having further desired properties such as, without limitation, long term storage at temperatures above freezing, injectable, moldable, rehydratable (such as at the time of use/administration), etc.

In an exemplary embodiment, the one or more isolated portions or layers of dermis (“isolated dermal tissue”) are then minimally processed to reduce and avoid unnecessary damage to the collagen structure of the skin sample. For example, without limitation, the isolated dermal tissue may be decellularized such as by physically, chemically, or both physically and chemically, treating it with saline, water, a buffer, a hypertonic solution, one or more detergents or surfactants, and combinations thereof, where treating may include one or more of contacting, rinsing, soaking, agitating, and blending, the isolated dermal tissue with the aforesaid materials. Furthermore, and without limitation, the isolated dermal tissue may be gently disinfected such as by contacting, rinsing, soaking, and combinations thereof, the isolated dermal tissue with one or more disinfecting agents (e.g., peracetic acid, ethanol, propylene glycol, etc.).

Of course, the isolated dermal tissue may be washed or rinsed one or more times with sterile water, in between or after any such decellularizing and disinfecting treatments to remove residual processing chemicals. The disinfected and acellular dermal tissue may then be cut into rectangular-shaped sheets or any appropriate and desirable shape suitable for clinical uses. The tissue sheets may again be disinfected or sterilized, such as by treating with aqueous ethanol, and then packaged to provide a hydrated collagen matrix, i.e., the improved ADM.

The processing techniques performed on each of the isolated portions and layers of dermal tissue are selected to preserve, or minimize destruction, of the structure of the extracellular matrix of the isolated portions or layers of dermal tissue, each of which ultimately forms an improved ADM. Accordingly, when used as a graft material, the resulting improved ADM provides a framework capable of supporting cellular repopulation, vascularization, and tissue regeneration at the surgical site, as described above.

As discussed above, selection of which type of dermal tissue and, therefore, which layer or portion of a layer of a skin sample, is isolated by the presently described and contemplated method will depend on the desired properties for the resulting one or more ADMs and the known or expected properties of each type of dermal tissue present in the skin sample. Determination of the desired properties for the resulting one or more ADMs is generally within the ability of persons of ordinary skill in the relevant art based on the intended use of the ADM(s), as is selection of which types of dermal tissue, i.e., which layer or portion of a layer of the skin sample, for isolation.

With reference now to FIG. 4, the selection and nomenclature of reference features in the skin sample that facilitate determining where to make each of the two or more cuts into the skin sample, will now be discussed. The reticular dermis RD of any skin sample, regardless of donor or location on donor from which it is recovered, may be assessed and defined as located between a first reticular defining plane FRP and a second reticular defining plane SRP. The first reticular defining plane FRP lies entirely within the reticular dermis RD and is adjacent to the hypodermis H and substantially parallel to and distal from the papillary-reticular interface PRI. The second reticular defining plane SRP lies entirely within the reticular dermis RD and is distal to the hypodermis H and substantially parallel and proximate to the papillary-reticular interface PRI. Moreover, the first and second reticular defining planes FRP, SRP may be advantageously selected and identified to provide a maximum and substantially uniform initial reticular thickness IRT therebetween. As previously stated, the reference plane REF is selected to provide a reference for alignment of all planar cuts and is shown in FIG. 4 as being aligned with the papillary-reticular interface PRI.

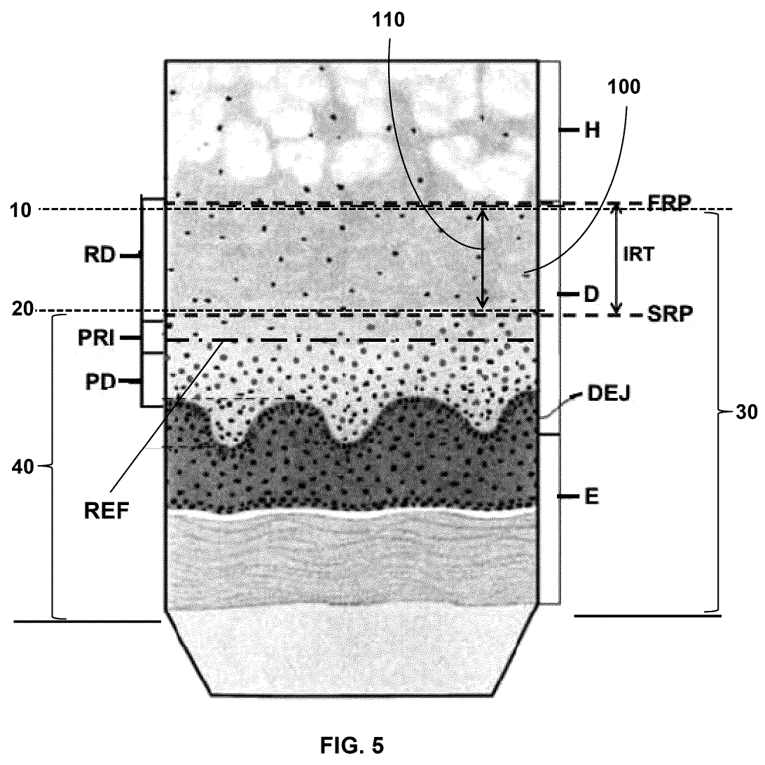

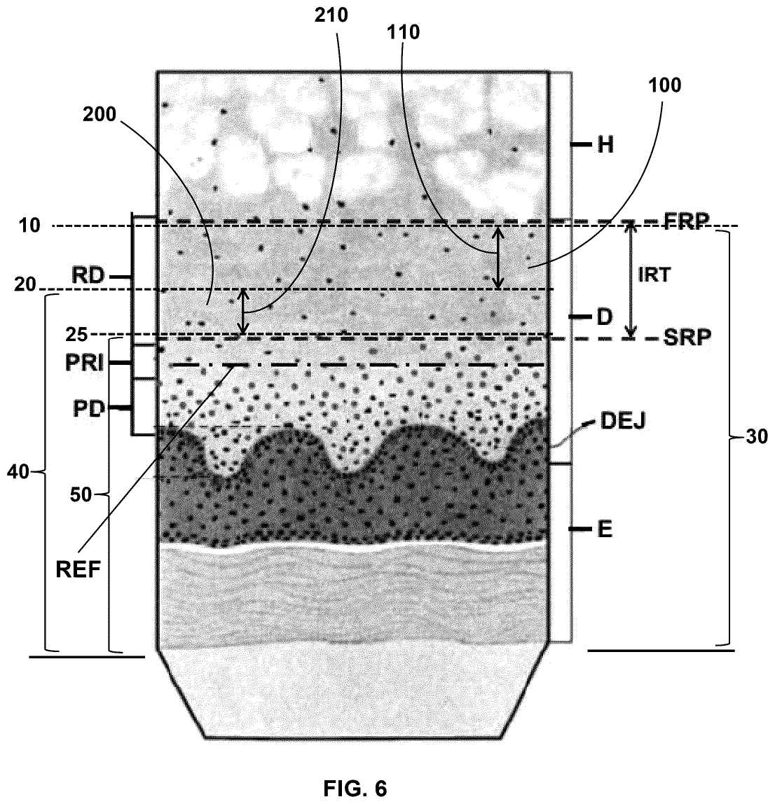

Prior to making cuts into the skin sample, the skin sample may be placed in an “epidermis face down” orientation (see, e.g., FIGS. 5 and 6), having its epidermis E face down at the bottom of the skin sample and in contact with a supporting surface (whether flat and planar such as a table or platform, or curved such as a drum or cylinder) upon which the skin sample is placed for processing. In the epidermis face down orientation of the skin sample, the reticular dermis RD (or hypodermis H if present) is face up at the top of the skin sample and remote from the supporting surface. Furthermore, when the skin sample is in the epidermis face down orientation and it is desired that the one or more improved ADMs consist essentially of reticular dermis RD, the two or more cuts are generally, but do not have to be made, from the top down, starting with the deeper portion of the reticular dermis RD.

However, the skin sample may alternatively be placed in an “epidermis face up” orientation (see, e.g., FIG. 1), i.e., opposite from the epidermis face down orientation described above and in which its epidermis E is face up at the top of the skin sample and remote from the supporting surface, while the reticular dermis RD (or hypodermis H if present) is face down at the bottom of the skin sample and potentially in contact with the supporting surface. Furthermore, when the skin sample is in the epidermis face up orientation and it is desired that the one or more improved ADMs consist essentially of reticular dermis RD, the two or more cuts are generally, but do not have to be made, in the order reverse to that stated above, from the top down, such as by starting with an upper portion of the reticular dermis RD.

Several more specific exemplary embodiments of methods for producing one or more improved ADMs from a skin sample will now be described, with reference to FIGS. 5-6.

In some embodiments, the method for producing an improved ADM, consisting essentially of reticular dermis RD, comprises the steps of:
providing a skin sample in an epidermis side down orientation and having at least the following layer-like components:
   (a) an epidermis E, (b) a dermis D comprising dermal tissue and underlying the epidermis, the dermis D including a papillary dermis PD adjacent the epidermis E, a reticular dermis RD distal to the epidermis E, and a papillary-reticular dermis interface (PRI) between the papillary dermis PD and reticular dermis RD, (c) a dermis-epidermis junction DEJ between the epidermis E and the papillary dermis PD; and (d) a hypodermis H comprising adipose tissue and underlying the reticular dermis RD, distal to the papillary-reticular dermis interface PRI,
   wherein the reticular dermis RD has an initial reticular thickness IRT defined between a first reticular defining plane FRP and a second reticular defining plane SRP, both of which are parallel to a reference plane REF which passes through the skin sample in an orientation approximately parallel with the papillary-reticular interface PRI,
making two planar cuts into the reticular dermis RD of the skin sample; isolating and recovering a single isolated portion of dermal tissue consisting essentially of reticular dermis RD; and performing one or more processing techniques on the single isolated portion of dermal tissue to produce the improved ADM which consists essentially of reticular dermis RD.

With reference to FIG. 5, a specific exemplary embodiment of the method for producing an improved ADM, consisting essentially of reticular dermis RD, will now be described in more detail. The steps of making two planar cuts and isolating and recovering a single isolated portion of dermal tissue comprise:
making a first planar cut 10 into the reticular dermis RD, proximate to or coextensive with the first reticular defining plane FRP, and parallel to the reference plane Ref;
removing the hypodermis H, and optionally a portion of the reticular dermis RD adjacent the hypodermis H, along the first planar cut to form a first exposed surface of a first remaining portion 30 of the skin sample which lacks the hypodermis H, and optionally a portion of the reticular dermis RD adjacent the hypodermis H;
making a second planar cut 20 parallel to the reference plane REF and into the reticular dermis RD of the first remaining portion of the skin sample, wherein the second planar cut is spaced from the first exposed surface by a first distance which is no greater than the initial reticular thickness IRT and is equal to a desired first ADM thickness 110; and removing and recovering a first isolated portion 100 of dermal tissue along the second planar cut 20 and leaving a second remaining portion 40 of the skin sample, thereby forming a second exposed surface on the first isolated portion 100 of dermal tissue, the second exposed surface being opposite the first exposed surface, wherein the first isolated portion 100 is the single isolated portion 100 of dermal tissue and has a substantially uniform thickness equal to the desired first ADM thickness 110.

Additionally, the method comprises applying one or more processing techniques, as discussed hereinabove, to the single isolated portion 100 of dermal tissue to produce the improved ADM which consists essentially of reticular dermis RD. The resulting improved ADM has one or more properties suitable for use of the improved ADM for breast reconstruction procedures.

As can be seen in FIG. 5, after performing the aforesaid cutting and removing steps, the second remaining portion 40 of the skin sample comprises at least the epidermis E, the dermis-epidermis junction DEJ, the papillary dermis PD, the papillary-reticular interface PRI, and optionally also a portion of reticular dermis adjacent to the papillary-reticular interface PRI, all of which may be discarded or repurposed.

Performing the foregoing method produces an improved ADM dermal tissue form for use in surgical procedures, comprising a portion of dermal tissue which consists essentially of reticular dermis and has a first exposed surface formed by a first cut into a reticular dermis of a skin sample and a second exposed surface opposite said first exposed surface and formed by a second cut into the reticular dermis of the skin sample, said portion of dermal tissue comprising a collagen matrix having substantially uniform density and porosity between said first exposed surface and said second exposed surface, and having a desired first allograft thickness between the first exposed surface and the second exposed surface.

In other exemplary embodiments, the method produces two or more improved ADMs. Such methods comprise cutting a skin sample three or more times to isolate and recover at least two portions of dermal tissue to form two or more independent ADMs, each comprising the same type of dermal tissue. Each of the at least two isolated portions of dermal tissue are subjected to one or more further processing techniques (in addition to the cutting already performed), which produces at least two separate improved ADMs, each of which comprises the same single dermal tissue type. The one or more further processing techniques to which each isolated portion of dermal tissue is subjected may be the same or different for each isolated portion of dermal tissue. For example, but without limitation, such a method may comprise performing three cuts, all of which are into the reticular dermis RD of the skin sample and produce two separate improved ADMs such as at least a portion of reticular dermis RD, from deeper within the same skin sample, whereby each of the resulting ADCMs essentially comprises reticular dermis RD. The procedure for preparing two or more such ADMs according to an exemplary embodiment of the invention is described below.

In some embodiments, the method for producing two or more improved ADMs, each consisting essentially of reticular dermis RD, comprises the steps of:
providing a skin sample in an epidermis side down orientation and having at least the following layer-like components:
   (a) an epidermis E, (b) a dermis D comprising dermal tissue and underlying the epidermis, the dermis D including a papillary dermis PD adjacent the epidermis E, a reticular dermis RD distal to the epidermis E, and a papillary-reticular dermis interface (PRI) between the papillary dermis PD and reticular dermis RD, (c) a dermis-epidermis junction DEJ between the epidermis E and the papillary dermis PD; and (d) a hypodermis H comprising adipose tissue and underlying the reticular dermis RD, distal to the papillary-reticular dermis interface PRI,
   wherein the reticular dermis RD has an initial reticular thickness IRT defined between a first reticular defining plane FRP and a second reticular defining plane SRP, both of which are parallel to a reference plane REF which passes through the skin sample in an orientation approximately parallel with the papillary-reticular interface PRI,
making at least three planar cuts into the reticular dermis RD of the skin sample; isolating and recovering a first isolated portion of dermal tissue and a second isolated portion of dermal tissue, each of which consists essentially of reticular dermis RD; and performing one or more processing techniques on each of the first and second isolated portions of dermal tissue to produce a first improved ADM and a second ACD, each of which consists essentially of reticular dermis RD.

With reference to FIG. 6, a specific exemplary embodiment of the method for producing at least two improved ADMs, each consisting essentially of reticular dermis RD isolated from the same skin sample, will now be described in more detail. The steps of making at least three planar cuts and isolating and recovering a first isolated portion of dermal tissue and a second isolated portion of dermal tissue comprise:
making a first planar cut 10 into the reticular dermis RD, proximate to or coextensive with the first reticular defining plane FRP, and parallel to the reference plane Ref;
removing the hypodermis H, and optionally a portion of the reticular dermis RD adjacent the hypodermis H, along the first planar cut to form a first exposed surface of a first remaining portion 30 of the skin sample which lacks the hypodermis H, and optionally a portion of the reticular dermis RD adjacent the hypodermis H;
making a second planar cut 20 parallel to the reference plane REF and into the reticular dermis RD of the first remaining portion 30 of the skin sample, wherein the second planar cut 20 is spaced from the first exposed surface by a first distance which is less than the initial reticular thickness IRT and is equal to a desired first ADM thickness 110;
removing and recovering a first isolated portion 100 of dermal tissue along the second planar cut 20 and leaving a second remaining portion 40 of the skin sample with a remaining portion of the initial reticular thickness IRT, thereby forming a second exposed surface on the first isolated portion 100 of dermal tissue and a third exposed surface on the second remaining portion 40 of the skin sample, wherein the second exposed surface is opposite the first exposed surface, wherein the first isolated portion 100 of dermal tissue has a substantially uniform thickness equal to the desired first ADM thickness 110; making a third planar cut 25 parallel to the reference plane REF and into the reticular dermis RD of the second remaining portion 40 of the skin sample, wherein the third planar cut is spaced from the third exposed surface by a second distance which is less than the remaining portion of the initial reticular thickness IRT and is equal to a desired second ADM thickness 210; and
removing and recovering a second isolated portion 200 of dermal tissue along the third planar cut 25 and leaving a third remaining portion 50 of the skin sample, thereby forming a fourth exposed surface on the second isolated portion 200 of dermal tissue, the fourth exposed surface being opposite the third exposed surface, wherein the first isolated portion 200 of dermal tissue a substantially uniform thickness equal to the desired second ADM thickness 210.

Since the first and second isolated portions of dermal tissue are produced from a single skin sample, the sum of the thickness of the first isolated portion and the thickness of the second isolated portion is no greater than the initial reticular thickness of the reticular dermis of the single skin sample. In some embodiments, the at least three cuts are made wherein the first thickness of the first isolated portion of dermal tissue is no more than about 80% of the initial reticular thickness and the second thickness of the second isolated portion of dermal tissue is at least 20% of the initial reticular thickness. These proportions are intended to ensure that the resulting second isolated portion of dermal tissue forms a second improved ADM which is suitable for use in soft tissue reconstruction and repair procedures. In some embodiments, the first thickness of the first isolated portion of dermal tissue is no more than about 70% of the initial reticular thickness and the second thickness of the second isolated portion of dermal tissue is at least 30% of the initial reticular thickness. In some embodiments, the first thickness of the first isolated portion of dermal tissue is about 50% of the initial reticular thickness and the second thickness of the second isolated portion of dermal tissue is about 50% of the initial reticular thickness.

Additionally, the method comprises applying one or more processing techniques, as discussed hereinabove, to each of the first and the second isolated portions 100, 200 of dermal tissue to produce a first improved ADM and a second improved ADM, each of which consists essentially of reticular dermis RD. The one or more processing techniques applies to each of the first isolated portion 100 of dermal tissue and the second isolated portion 200 of dermal tissue may be the same of different. Both of the resulting first and second improved ADMs have one or more properties, which may be the same or different, and are suitable for using each of the first and second improved ADMs for breast reconstruction procedures.

As can be seen in FIG. 6, after performing the aforesaid first cutting and removing steps, the first remaining portion 30 of the skin sample remains and comprises at least the epidermis E, the dermis-epidermis junction DEJ, the papillary dermis PD, the papillary-reticular interface PRI, and reticular dermis RD. Furthermore, after performing the aforesaid second cutting and removing steps, the second remaining portion 40 of the skin sample remains and comprises at least the epidermis E, the dermis-epidermis junction DEJ, the papillary dermis PD, the papillary-reticular interface PRI, and a portion of reticular dermis RD adjacent the papillary-reticular interface PRI and having the remaining portion of the initial reticular thickness IRT. Finally, after performing the aforesaid third cutting and removing steps, the third remaining portion 50 of the skin sample remains and comprises at least the epidermis E, the dermis-epidermis junction DEJ, the papillary dermis PD, the papillary-reticular interface PRI, and optionally also a portion of reticular dermis adjacent to the papillary-reticular interface PRI, all of which may be discarded or repurposed.

Performing the foregoing method produces two or more improved ADM dermal tissue forms for use in surgical procedures, each comprising an isolated portion (e.g., layer or sheet) of dermal tissue which consists essentially of reticular dermis. The first improved ADM comprises a first isolated portion of dermal tissue and has a first exposed surface formed by a first cut into a reticular dermis of a skin sample and a second exposed surface opposite said first exposed surface and formed by a second cut into the reticular dermis of the skin sample.

The second improved ADM comprises a second isolated portion of dermal tissue and has a third exposed surface formed by a second cut into a reticular dermis of a skin sample and a fourth exposed surface opposite said first exposed surface and formed by a third cut into the reticular dermis of the skin sample. The first and second isolated portions (e.g., layers, sheets) of dermal tissue each, independently of the other, consists essentially of reticular dermis RD and also comprises a collagen matrix having substantially uniform density and porosity between their relative exposed surfaces.

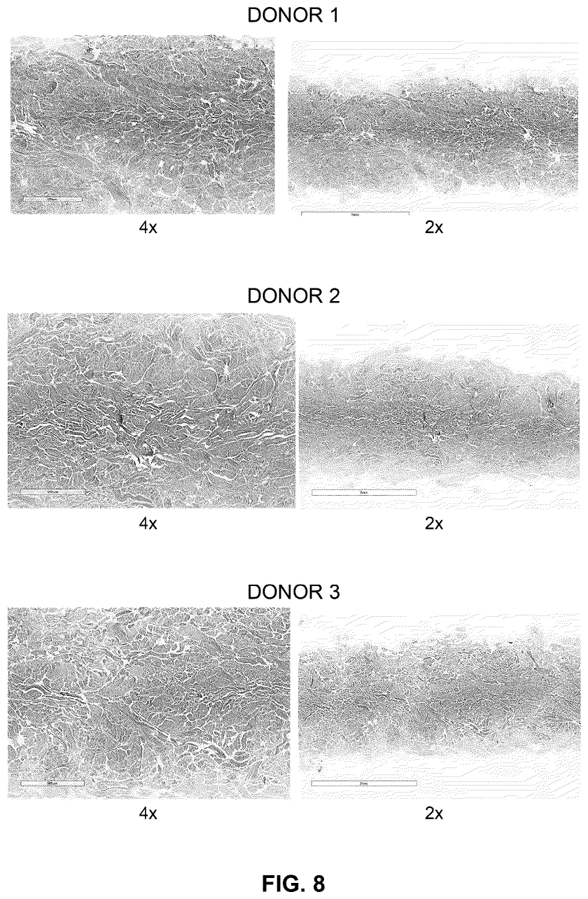

FIG. 8 provides cross-sectional histology images (i.e., hematoxylin and eosin stained, “H&E”, having magnification 4× and 2×) of three exemplary improved ADMs produced from skin samples recovered from three different donors. Each skin sample was processed by making three cuts, according to the method described above with reference to FIG. 6, thereby isolating and recovering two individual portions of dermal tissue, each of which formed an improved ADM consisting essentially of reticular dermis. It will be understood that performing the method of making three cuts into each of three skin samples produced a total of six improved ADMs, each consisting essentially of reticular dermis, and FIG. 8 provides histology images for three of them, i.e., one improved ADM from each pair produced from a single skin sample. As shown in the histology images of FIG. 8, each of the three improved ADMs had uniform cross-sectional density and porosity.

It will be understood that any and all of the above-described methods for producing one or more improved ADMs may be performed by providing a skin sample in an epidermis side up orientation (e.g., see FIGS. 1 and 4) and then making cuts and removing and recovering one or more isolated portions of dermal tissue would be performed in the opposite order as delineated above. For example, without limitation, making a first planar cut into the reticular dermis RD, proximate to or coextensive with the second reticular defining plane FRP, and parallel to the reference plane REF, followed by removing the epidermis E, the dermis-epidermis junction DEJ, the papillary dermis PD, the papillary-reticular dermis interface PRI, and optionally a portion of the reticular dermis RD adjacent the papillary-reticular dermis interface PRI, from the skin sample, which would leave a remaining portion of the skin sample. The remaining portion of the skin sample would comprise a portion of reticular dermis RD having a thickness approximately equal to or less than the initial reticular thickness IRT, and also, if present, the hypodermis H.

Such a method which starts with the skin sample in an epidermis side up orientation would then comprise making a first planar cut into the reticular dermis RD parallel to the reference plane REF, wherein the first planar cut would be remote from the first reticular defining plane FRP, and optionally proximate to or coextensive with the second reticular defining plane SRP. Making the first planar cut would be followed by removing the epidermis E, the dermis-epidermis junction DEJ, the papillary dermis PD, the papillary-reticular dermis interface PRI, and optionally a portion of the reticular dermis RD adjacent the papillary-reticular dermis interface PRI, from the skin sample, which would leave a remaining portion of the skin sample. The remaining portion of the skin sample would comprise a portion of reticular dermis RD and if present, the hypodermis H. The portion of reticular dermis RD would have a first exposed surface and a thickness approximately equal to or less than the initial reticular thickness IRT.

This embodiment of the method, with the skin sample in the epidermal face up orientation, would further comprise making a second planar cut parallel to the reference plane REF and into the reticular dermis RD of the remaining portion of the skin sample. The second planar cut is spaced from the first exposed surface by a first distance which is equal to a desired first ADM thickness. Furthermore, the second cut may or may not be proximate or coextensive with the first reticular defining plane FRP.

Making the second cut would be followed by removing and recovering a first isolated portion of dermal tissue along the second planar cut. The hypodermis H would remain as well as, optionally, a second remaining portion the reticular dermis RD adjacent thereto and having a remaining reticular thickness. Removing the first isolated portion of dermal tissue would form a second exposed surface on the first isolated portion of dermal tissue, as well as a third exposed surface on the subsequent remaining portion of the skin sample. The second exposed surface of the first isolated portion of dermal tissue is opposite the first exposed surface, and the first isolated portion of dermal tissue has a substantially uniform thickness equal to the desired first ADM thickness between the first and second exposed surfaces. The first isolated portion of dermal tissue is next subjected to one or more processing techniques, to produce a first improved ADM.

Depending on how much reticular dermis RD remains on the hypodermis H which remains after the first and second cuts are made and the first isolated portion of dermal tissue is removed, additional cuts into the reticular dermis RD may be made to produce additional isolated portion of dermal tissue, which may be also processed to produce another one or more improved ADMs.

It will be understood that the embodiments described herein are merely exemplary and that a person of ordinary skill in the art may make many variations and modifications without departing from the spirit and scope of the invention. All such variations and modifications are intended to be included within the scope of the invention, and the appended claims. Some of the possible variations and modifications of the improved ADM and the dermis/adipose hybrid bilayer tissue form are disclosed below.

In other embodiments, the improved ADMs may be provided in perforated or meshed form. Perforating the improved ADMs or forming a mesh of the improved ADM makes it more porous, and ideal for certain surgical applications.

In other embodiments, the improved ADMs may be further processed by using known methods to reduce the dimensions (size and shape) of the improved ADMs, thereby producing a particulate improved ADM graft comprising ADM particles, ADM fibers, or both.

In some embodiments, post-processing comprises combining the particulate improved ADM with one or more carriers. Suitable carriers include, without limitation, at least one of an isotonic solution, a sodium chloride solution, lactated Ringer's solution, a phosphate-buffered saline solution (PBS), platelet rich plasma (PRP), hyaluronic acid (HA) or a derivative thereof such as sodium hyaluronate. In some embodiments, the carrier is a sodium chloride solution at a concentration of about 0.1% to about 1%. In some such embodiments, the sodium chloride solution is at a concentration of about 0.9%. In some embodiments, the carrier is a mixture of sodium hyaluronate and an aqueous solution. In some embodiments, the sodium hyaluronate has a molecular weight of from about 5.0×10³ to about 3.0×10⁶ Daltons, such as from about 6.0×10⁵ to about 3.0×10⁶ Daltons and is mixed with an aqueous solution to form a matrix-carrier mixture having a viscosity ranging from about 1000 centipoise to about 275,000 centipoise, such as from about 6,000 to about 275,000 centipoise. In some embodiments, the aqueous solution of the carrier comprising sodium hyaluronate is, for example without limitation, one or more of water, saline, phosphate buffered solution (PBS), isotonic saline, and the like.

In some embodiments, the carrier comprises thrombin, fibrin, or a combination thereof. In some embodiments, the carrier comprises glycerin. In some embodiments, the carrier comprises collagen. In some embodiments, the carrier comprises lecithin. In some embodiments, the carrier comprises a sugar. In some embodiments, the carrier comprises a polysaccharide. In some embodiments, post-processing comprises mixing the particulate improved ADM with a carrier on site for immediate administration to a patient.

In some embodiments, the particulate improved ADM is mixed or otherwise combined with a carrier such that the particulate improved ADM and carrier combine to form a flowable gel. In some embodiments, mixing with a carrier is performed at a pH in the range of from about 2 to about 8. In some embodiments, mixing with a carrier is performed at a pH in the range of about 4 to about 8. In some embodiments, mixing with a carrier is performed at a pH below the isoelectric point of collagen.

In some embodiments, the particulate improved ADM is mixed or otherwise combined with a carrier such that the particulate improved ADM and the carrier form a paste. In some embodiments, the carrier includes a polymer such that the particulate improved ADM and the polymer form a paste. In some embodiments, the polymer is selected from the group consisting of polysaccharides, nucleic acids, carbohydrates, proteins, polypeptides, poly(α-hydroxy acids), poly(lactones), poly(amino acids), poly(anhydrides), poly(orthoesters), poly(anhydride-co-imides), poly(orthocarbonates), poly(α-hydroxy alkanoates), poly(dioxanones), poly(phosphoesters), poly(L-lactide) (PLLA), poly(D,L-lactide) (PDLLA), polyglycolide (PGA), poly(lactide-co-glycolide (PLGA), poly(L-lactide-co-D, L-lactide), poly(D,L-lactide-co-trimethylene carbonate), polyhydroxybutyrate (PHB), poly(ε-caprolactone), poly(δ-valerolactone), poly(γ-butyrolactone), poly(caprolactone), polyacrylic acid, polycarboxylic acid, poly(allylamine hydrochloride), poly(diallyldimethylammonium chloride), poly(ethyleneimine), polypropylene fumarate, polyvinyl alcohol, polyvinylpyrrolidone, polyethylene, polymethylmethacrylate, carbon fibers, poly(ethylene glycol), poly(ethylene oxide), poly(vinyl alcohol), poly(vinylpyrrolidone), poly(ethyloxazoline), poly(ethylene oxide)-co-poly(propylene oxide) block copolymers, poly(ethylene terephthalate) polyamide, copolymers thereof, and combinations thereof.

In some embodiments the carrier includes a glycerin and/or glycerol. In some embodiments the carrier includes gelatin. In some embodiments the carrier includes alginates. In some embodiments the carrier includes an isotonic solution. In some embodiments the carrier includes a sodium chloride solution. In some embodiments the carrier includes a lactated Ringer's solution. In some embodiments the carrier includes a phosphate-buffered saline. In some embodiments the carrier includes platelet rich plasma. In some embodiments the carrier includes a hyaluronic salt. In some embodiments the carrier includes a hyaluronic ester According to some embodiments the carrier includes fibrin. In some embodiments the carrier includes thrombin. In some embodiments the carrier includes a collagen. In some embodiments the carrier includes lecithin. In some embodiments the carrier includes a combination of two or more carrier components.

In other embodiments, cells may be added to the improved ADMs. Such cells may include, for example, stem cells (e.g., embryonic stem cells, mesenchymal stem cells, adult stem cells, skin-derived stem cells, and amnion-derived stem cells), fibroblasts, osteoblasts, myoblasts, adipocytes, and keratinocytes.

In other embodiments, biological substances may be added to the improved ADM. Such biological substances may include, for example, platelet-rich plasma (“PRP”), bone marrow aspirate, lipoaspirate, and/or demineralized bone particles or fibers and/or other allograft tissue forms. Further, one or more other tissue derived matrices may be added or combined with the ADMs. For example, without limitation, amnion tissue (with or without the native cells thereof) may be added to the Disclosed ADM, e.g., to function as an anti-adhesion membrane.

In some embodiments, the improved ADM may, regardless of physical form, be hydrated or, if partially or fully dehydrated, then rehydrated, by addition of, or combination with, a biocompatible fluid or carrier. Such hydrating or rehydrating may be performed at any time after the cutting steps are performed to produce at least one isolated portion of dermal tissue useful as the improved ADM for example, but not limited to, during post-processing, or in preparation for use and administration. Fully dehydrated, as used herein, means containing less than about 5% water, by weight, based on the total weight of the improved ADM.

In other embodiments, the improved ADMs may be used to wrap around the above-identified biological substances or other biological substances. In such a wrapper function, the improved ADMs may protect, enclose, and or insulate such biological substances upon implantation.

In other embodiments, reinforcing elements may be added to the improved ADM. Examples of such reinforcing elements include absorbable fibers and non-absorbable fibers. The reinforcing elements may be arranged in various patterns, such as, for example, a grid pattern.

In other embodiments, the improved ADM may be chemically modified to imbue it with enhanced properties. One example is cross-linking the collagen of the improved ADM.

### EXAMPLES

Skin samples (which may or may not have include full thickness skin) were obtained and cut according to the methods described above with reference to the diagrams provided in FIGS. 5 and 6, to produce several isolated portions of dermal tissue, each of which consisted essentially of reticular dermis from the skin samples. Each of the isolated portions of dermis was then processed, as described in Example 1 below, to produce several improved ADM grafts produced by the methods shown in FIGS. 5 and 6, which were then subjected to histological analysis as further described below in Example 2.

### Example 1-Dermis Processing Performed after Dermis Sample is Cut

### General Dermis Processing Procedure—Incoming to Packaging

1. Each cut isolated portion of dermis tissue (“isolated portion”) was decellularized by:
a. soaking in a sodium chloride solution, followed by 2 water rinses, wherein after the first rinse, the water is replaced with fresh water for performing the subsequent second rinsing step,
b. then the rinsed isolated portions were soaked in a Triton type of nonionic surfactant/detergent, followed by 8 water rinses, in between each of which the rinse water is replaced with fresh water for performing the subsequent rinsing, and
c. in between the foregoing soaking and rinsing steps, additional unwanted components or features visible by eye were removed from the isolated portions.

2. The decellularized and rinsed isolated portions were then disinfected by:
a. soaking in a peracetic acid solution (aqueous), under pressure, followed by 8 water rinses, in between each of which the rinse water is replaced with fresh water for performing the subsequent rinsing.

3. The disinfected and rinsed isolated portions were then cut to desired size and shape and placed in ethanol (aqueous solution) for storage and/or packaging (in ethanol) in a triple bag configuration of foil and 2 Tyvek pouches (TYVEK is a trademark of DuPont of Wilmington, Delaware, U.S.A.).

### Example 2—Histological Analysis of Cut and Processed Improved ADM Grafts

1. Each improved ADM graft, consisting essentially of reticular dermis, was cut to produce 1 cm×1 cm coupons/pieces, which were rinsed by submerging in saline to remove ethanol.

2. At least one coupon/piece from each improved ADM graft was placed in neutral buffer formalin or equivalent fixative, and shipped to a lab for histology.

3. At the lab each fixed coupon/piece was blocked in paraffin, then sliced to create a thin cross section of tissue, and stained with H&E (hematoxylin and eosin) in a standard and well understood procedure. As a result of the H&E staining, the collagen matrix was stained pink and, and cellular nuclei were stained purple. This allowed observed and visualization of the collagen structure of decellularized improved ADM grafts.

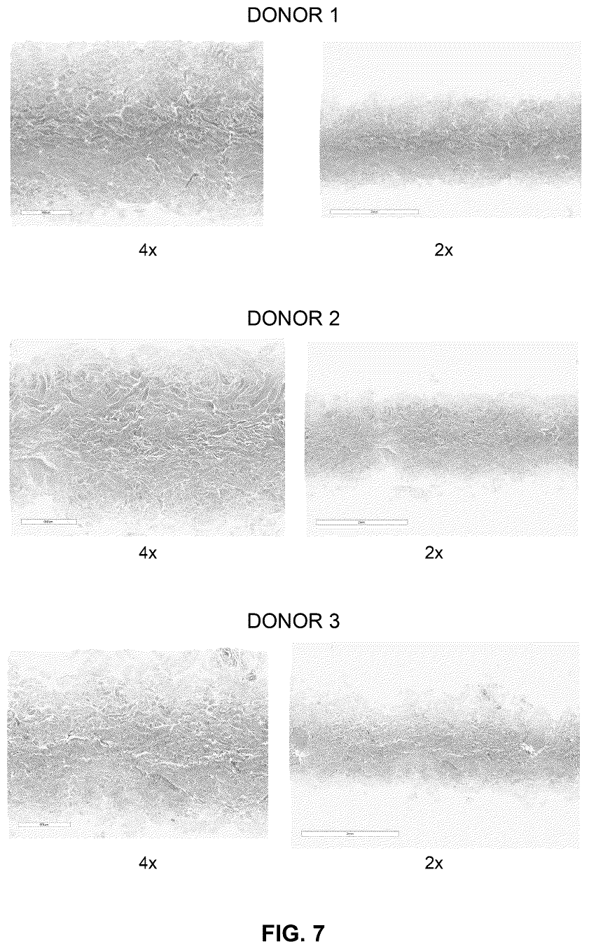

4. FIG. 7 provides histology images converted to black and white (grey scale), at magnification 4× and 2×, and each produced with saturation of 4000% and sharpness of 50%) of coupons/pieces taken from three improved ADMs produced from skin samples recovered from three different donors, each improved ADM having uniform cross-sectional density and porosity and produced by a method comprising cutting a skin sample two times as shown in FIG. 5.

5. FIG. 8 provides histology images converted to black and white (grey scale), at magnification 4× and 2×, each produced with saturation of 4000% and sharpness of 50%) of three improved ADMs produced from skin samples recovered from three different donors, each improved ADM having uniform cross-sectional density and porosity, and being one of a pair of improved ADMs produced from a single skin sample by a method comprising cutting the skin sample three times as shown in FIG. 6.

We claim:

1. A dermal tissue form which is useful in surgical procedures and is derived from a donor skin tissue which comprised (a) an epidermis; (b) a dermis underlying the epidermis, the dermis including a papillary dermis adjacent the epidermis, a reticular dermis distal to the epidermis, a papillary-reticular dermis interface between the papillary dermis and reticular dermis; (c) and optionally a hypodermis underlying the reticular dermis of (b) the dermis and containing adipose tissue, wherein the reticular dermis has an initial reticular thickness defined between a first reticular defining plane and a second reticular defining plane, each of which is parallel to a reference plane which passes through the skin sample in an orientation approximately parallel with the papillary-reticular interface;
the dermal tissue form consisting essentially of reticular dermis and comprising: a first exposed surface formed by reticular dermis, an opposite second exposed surface formed by reticular dermis, a desired thickness between the first and second exposed surfaces, and uniform density and uniform porosity between the first and second exposed surfaces,
wherein the dermal tissue form promotes rapid and efficient cellular ingrowth and tissue ingrowth substantially equally from either the first or second exposed surfaces upon implantation; and
wherein the dermal tissue form essentially lacks an epidermis, a papillary dermis, papillary-reticular dermis interface, a portion of the reticular dermis attached to the papillary-reticular dermis interface, the hypodermis if initially present on the skin sample, and a portion of the reticular dermis attached to the hypodermis.

2. The dermal tissue form of claim 1, wherein said dermal tissue form is perforated.

3. The dermal tissue form of claim 1, wherein said dermal tissue form is in mesh form.

4. The dermal tissue form of claim 1, wherein said dermal tissue form is in particulate form and comprises particles, fibers, or both.

5. The dermal tissue form of claim 1, further comprising cells.

6. The dermal tissue form of claim 1, further comprising one or more biological substances.

7. A pair of dermal tissue forms, each of which is useful in surgical procedures and consists essentially of reticular dermis, and both of which are derived from a single donor skin tissue which comprised (a) an epidermis; (b) a dermis underlying the epidermis, the dermis including a papillary dermis adjacent the epidermis, a reticular dermis distal to the epidermis, a papillary-reticular dermis interface between the papillary dermis and reticular dermis; (c) and optionally a hypodermis underlying the reticular dermis of (b) the dermis and containing adipose tissue, wherein the reticular dermis has an initial reticular thickness defined between a first reticular defining plane and a second reticular defining plane, each of which is parallel to a reference plane which passes through the skin sample in an orientation approximately parallel with the papillary-reticular interface;
wherein a first dermal tissue form of the pair comprises: a first exposed surface formed by reticular dermis, an opposite second exposed surface formed by reticular dermis, a first desired thickness between the first and second exposed surfaces, and uniform density and uniform porosity between the first and second exposed surfaces,
wherein a second dermal tissue form of the pair comprises: a third exposed surface formed by reticular dermis, an opposite fourth exposed surface formed by reticular dermis, a second desired thickness between the third and fourth exposed surfaces, and uniform density and uniform porosity between the third and fourth exposed surfaces;
wherein each of the pair of dermal tissue forms promotes rapid and efficient cellular ingrowth and tissue ingrowth substantially equally from either the first or second exposed surfaces upon implantation; and
wherein each of the pair of dermal tissue forms essentially lacks an epidermis, a papillary dermis, papillary-reticular dermis interface, a portion of the reticular dermis attached to the papillary-reticular dermis interface, the hypodermis if initially present on the skin sample, and a portion of the reticular dermis attached to the hypodermis.

8. The pair of dermal tissue forms of claim 7, wherein at least one of the first dermal tissue and the second dermal tissue form is perforated.

9. The pair of dermal tissue forms of claim 7, wherein at least one of the first dermal tissue and the second dermal tissue form is in mesh form.

10. The pair of dermal tissue forms of claim 7, wherein at least one of the first dermal tissue and the second dermal tissue form is in particulate form and comprises particles, fibers, or both.

11. The pair of dermal tissue forms of claim 7, wherein at least one of the first dermal tissue and the second dermal tissue form further comprises cells.

12. The pair of dermal tissue forms of claim 7, wherein at least one of the first dermal tissue and the second dermal tissue form further comprises one or more biological substances.

13. A method for producing at least a pair of dermal tissue forms, comprising at least a first dermal tissue form and a second dermal tissue form, each of which is useful for soft tissue repair in surgical procedures and consists essentially of reticular dermis, and all of which are derived from a single donor skin sample, the method comprising the steps of:
providing a donor tissue including a skin sample having:
(a) an epidermis;
(b) a dermis underlying the epidermis, the dermis including a papillary dermis adjacent the epidermis, a reticular dermis distal to the epidermis, and a papillary-reticular dermis interface between the papillary dermis and reticular dermis; and
(c) optionally a hypodermis underlying the reticular dermis and containing adipose tissue, wherein the reticular dermis has an initial reticular thickness defined between a first reticular defining plane and a second reticular defining plane, each of which is parallel to a reference plane which passes through the skin sample in an orientation approximately parallel with the papillary-reticular interface;
making at least three planar cuts, which comprise a first planar cut, a second planar cut, and a third planar cut, each of which is made into the reticular dermis of the skin sample and is substantially parallel to other planar cuts, or substantially parallel to the reference plane, or both;
isolating and recovering a first isolated portion of dermal tissue having a first thickness between a first exposed surface and an opposite second exposed surface thereof, and a second isolated portion of dermal tissue having a second thickness between a third exposed surface and an opposite fourth exposed surface thereof, each of the first isolated portion and the second isolated portion consists essentially of reticular dermis, wherein the sum of the first thickness and the second thickness is no greater than the initial reticular thickness of the reticular dermis of the single skin sample; and
producing:
the first dermal tissue form using the first isolated portion of dermal tissue, wherein the first dermal tissue form has a desired first thickness equal to the first thickness of the first isolated potion of dermal tissue, and
the second dermal tissue form using the second isolated portion of dermal tissue, wherein the second dermal tissue form has a desired second thickness equal to the second thickness of the second isolated potion of dermal tissue.

14. The method of claim 13, wherein the first thickness of the first isolated portion of dermal tissue is no more than about 80% of the initial reticular thickness and the second thickness of the second isolated portion of dermal tissue is at least 20% of the initial reticular thickness.

15. The dermal tissue form of claim 13, further comprising selecting and identifying the reference plane of the skin sample to provide a reference for selecting, identifying, and alignment of the first and second reticular defining planes and all planar cuts with one another and the papillary-reticular interface.

16. The method of claim 13, further comprising selecting and identifying the first reticular defining plane and the second reticular defining plane to provide a maximum and substantially uniform initial reticular thickness therebetween.

17. The method of claim 13, wherein the desired first thickness of the first dermal tissue form is no more than about 80% of the initial reticular thickness and the desired second thickness of the second dermal tissue form is at least about 20% of the initial reticular thickness.

18. The method of claim 13, wherein making the first planar cut into the reticular dermis forms a first exposed surface of the first dermal tissue form, making the second planar cut into the reticular dermis forms a second exposed surface, opposite the first exposed surface, of the first dermal tissue form and, further, forms a third exposed surface of the second dermal tissue from, and making the third planar cut into the reticular dermis forms a fourth exposed surface, opposite the third exposed surface of the second dermal tissue form, wherein each of the first, second, third, and fourth exposed surfaces consist essentially of reticular dermis.

19. The method of claim 13, wherein making the second planar cut comprises selecting a location in the reticular dermis spaced apart from the first planar cut by a first distance equal to the first thickness of the first isolated portion of dermal tissue and no more than about 80% of the initial reticular thickness, and
making the third planar cut comprises selecting a location in the reticular dermis spaced apart from the first planar cut by a second distance equal to the second thickness of the second isolated portion of dermal tissue and at least about 20% of the initial reticular thickness, wherein a sum of the first thickness and the second thickness are no more than the initial reticular thickness.

20. The method of claim 13, wherein the skin sample is positioned in an epidermis face down orientation prior to the step of making at least three planar cuts which comprises:
making the first planar cut into the reticular dermis, proximate to or coextensive with the first reticular defining plane, and parallel to the reference plane;
removing the hypodermis H when present on the skin sample;
removing a portion of the reticular dermis which is either adjacent the hypodermis if present or most remote from the epidermis, along the first planar cut to form the first exposed surface of a first remaining portion of the skin sample which lacks the hypodermis and, optionally, a portion of the reticular dermis adjacent the hypodermis;
making the second planar cut parallel to the reference plane and into the reticular dermis of the first remaining portion of the skin sample, wherein the second planar cut is spaced from the first exposed surface by a first distance which is less than the initial reticular thickness and equal to the first thickness of the first isolated portion of dermal tissue;
removing and recovering the first isolated portion of dermal tissue along the second planar cut, thereby forming the second exposed surface on the first isolated portion of dermal tissue, and leaving a second remaining portion of the skin sample with a remaining portion of the initial reticular thickness and having the third exposed surface on the second remaining portion of the skin sample, wherein the first isolated portion of dermal tissue has a substantially uniform thickness equal to the desired first thickness of the first dermal tissue form;
making the third planar cut parallel to the reference plane and into the reticular dermis of the second remaining portion of the skin sample, wherein the third planar cut is spaced from the third exposed surface by a second distance which is less than the remaining portion of the initial reticular thickness and is equal to the desired second thickness of the second dermal tissue form; and
removing and recovering the second isolated portion of dermal tissue along the third planar cut and leaving a third remaining portion of the skin sample, thereby forming the fourth exposed surface on the second isolated portion of dermal tissue, wherein the first isolated portion of dermal tissue a substantially uniform thickness equal to the desired second thickness of the first dermal tissue form.
